SEQUENCE LISTING (8 sequences)

SEQ ID NO: 1 - DNA, 20 bp, Artificial Sequence
  primer
ccaaatctgtctccccagaa

SEQ ID NO: 2 - DNA, 20 bp, Artificial Sequence
  primer
tcaaaaacgaaggggagatg

SEQ ID NO: 3 - DNA, 20 bp, Artificial Sequence
  primer
ccatgtccacacaaggacag

SEQ ID NO: 4 - DNA, 20 bp, Artificial Sequence
  primer
gccagagtggctttctcaac

SEQ ID NO: 5 - DNA, 20 bp, Artificial Sequence
  primer
gcttgaaaagggttgagcag

SEQ ID NO: 6 - DNA, 20 bp, Artificial Sequence
  primer
ttgagtcccggtagaccaac

SEQ ID NO: 7 - DNA, 20 bp, Artificial Sequence
  primer
gaggcctggactctcaactg

SEQ ID NO: 8 - DNA, 20 bp, Artificial Sequence
  primer
aatgaatgggggttgaatga

(19) United States
(12) Patent Application Publication
(10) Pub. No.: US 2023/0148647 A1
(43) Pub. Date: May 18, 2023

(54) COMPOSITION FOR PROMOTING BIOSYNTHESIS OF ELASTIN AND COLLAGEN IN CONNECTIVE TISSUE

(71) Applicant: ELASTIC LAB INC., Gwangju (KR)

(72) Inventor: Manseok KIM, Seoul (KR)

(21) Appl. No.: 17/259,903

(22) PCT Filed: Oct. 8, 2020
(86) PCT No.: PCT/KR2020/013780

§ 371 Date: Jan. 12, 2021

(30) Foreign Application Priority Data
Sep. 2, 2020 (KR) 10-2020-0111559

(51) Int. Cl. A23L 33/175; A61K 31/198; A61K 31/401; A61K 31/728; A61K 33/06; A61K 33/24; A61K 33/30; A61K 33/32; A61K 33/34; A61K 8/27; A61K 8/44; A61P 19/02; A61P 21/00; A61P 9/12; A61Q 19/08

(52) U.S. Cl. CPC A23L 33/175; A61K 31/198; A61K 31/401; A61K 31/728; A61K 33/06; A61K 33/24; A61K 33/30; A61K 33/32; A61K 33/34; A61K 8/27; A61K 8/447; A61P 19/02; A61P 21/00; A61P 9/12; A61Q 19/08

(57) ABSTRACT

Disclosed is a composition for promoting biosynthesis of elastin or collagen. The composition improves the ability of fibroblasts to biosynthesize elastin and collagen and is thus used to restore skin elasticity deteriorated due to aging or light exposure, remove wrinkles and facial lines, improve vascular elasticity deteriorated due to aging, and hypertension and decreased blood circulation caused thereby, and enhance tendon and ligament elasticity and joint elasticity.

### BACKGROUND OF THE INVENTION

### Field of the Invention

The present invention relates to a composition for promoting the biosynthesis of elastin and/or collagen, and more specifically to a composition for promoting biosynthesis of elastin or collagen containing glycine, L-proline and divalent metal ions.

### Description of the Related Art

Elastin is a protein that is present in connective tissue along with collagen and has elasticity like rubber, and is involved in tissue flexibility and elasticity, can return to the original shape thereof even after being stretched or shrunk due to these properties, and plays an important role in skin elasticity, wrinkle prevention, contraction and relaxation of blood vessels, contraction and relaxation of the lungs, and the like.

Elastin fibers consist of elastin and fibrillin, which are composed of simple amino acids such as glycine, valine, alanine and proline. Elastin is produced through binding between a number of tropoelastins. The amino acids constituting elastin are hydrophobic amino acids including amino acids such as glycine and proline. These amino acids are cross-linked with lysine residues to form hydrophobic regions that can move freely.

Fibroblasts, endothelial cells, smooth muscle cells, and chondrocytes are responsible for the biosynthesis of elastin in vivo. Elastin can be stretched up to 7 times the length thereof, return to the original dimensional modulus thereof without significant molecular deformation, and theoretically repeat this stretching and restoration indefinitely.

Meanwhile, collagen is a major tissue-forming protein that fills the extracellular area of various connective tissues in the animal's body. Collagen is also the most abundant protein that accounts for 25% to 35% of all protein in mammals. Depending on the degree of mineralization, collagen may be hard like bone, may be flexible like tendons, or may range from hard to flexible like cartilage. Collagen takes the form of very long fibrils, is commonly found in fibrous tissues such as the tendons, ligaments and skin, and is also found in the cornea, cartilage, bones, blood vessels, digestive tract, intervertebral discs, and dentin of teeth. Collagen is a major component of the endomysium in muscle tissue. Collagen accounts for 1 to 2% of muscle tissue and 6% of strong muscles with many tendons. Fibroblasts, which are the most common cells in the body, produce and secrete collagen.

Skin aging and photoaging cause wrinkles on the skin surface, and the network of elastin fibers located in the distal part (reticular dermis) of the dermal tissue decreases, resulting in a significant loss of overall elasticity of the skin, eventually reducing the ability of the skin connective tissue to adapt to mechanical stretching and causing tissue sagging and loss of skin elasticity.

The gene (ELN-gene) encoding the protein tropoelastin, which is a precursor of elastin, encodes various forms of tropoelastin, and the gene begins to be expressed in the foetal stage, and remains active during the first 5 years of life, and then the activity thereof slows rapidly and ceases (Bashir, M M et al., J. Biol. Chem. 264:8887, 1989). That is, synthesis of the elastic elements of connective tissue, particularly the dermis, mucous membrane, cartilage tissue, endovascular membrane, lung and valve/myocardial connective tissue, ceases in an early stage of life. Replenishment does not occur except in cases where severe tissue damage such as burns or severe photoaging occurs, and such cases involve, for example, the overexpression of LOX (lysine oxidase) genes, which encode five different enzymes that catalyze oxidization of lysine residues among tropoelastin precursor molecules, and such expression is a stage required for the synthesis of functional elastin and subsequent introduction thereof into microfibrils that adhere to the cell surface. In particular, the enzyme-dependent process consists of lysine oxidation and simultaneous formation of Schiff bases between the amino groups of L-LYS and aldose to form crosslinked intramolecular bonds (Maki et al., Am. J. Pathol. 167:927, 2005). Elastin is the only connective tissue protein that is virtually irreplaceable, and remains the same for more than 70 years (having an average half-life of 74 years).

Meanwhile, type I collagen is a collagen that accounts for the largest proportion of human collagen, is a protein that is found in various areas such as the skin, blood vessels, organs and bones, and plays an important role in the organization of the extracellular matrix (ECM), and structural and functional maintenance in connective tissue. Collagen I deficiency is closely related to the loss of trophism and elasticity of human skin, especially in the phenomenon of skin degeneration typical of skin aging and photoaging.

Certain amino acids and oligopeptides are known to promote gene expression causing the biosynthesis of dermal and epidermal connective tissues, particularly, collagen and tropoelastin, when appropriately delivered and applied topically or orally (Lupo M P et al., Cosmeceutical peptides. Dermatol. Ther. 20:343, 2007), and topical or oral compositions using an amino acid mixture containing glycine, proline, alanine, valine, leucine and lysine hydrochloride mixed in an appropriate ratio have been disclosed, but promoting the synthesis of collagen and elastin has not been disclosed (see WO 2016/088078A, EP 2033689A and WO 2007/048522A, etc.).

Therefore, as a result of extensive efforts to develop a composition containing an optimal amino acid that is capable of promoting the biosynthesis of elastin and collagen, the present inventors found that, when treating fibroblasts with a composition containing glycine, proline, and divalent metal ions, the expression levels of elastin and type I collagen were increased. The present invention has been completed based on this finding.

### SUMMARY OF THE INVENTION

It is an object of the present invention to provide a composition for promoting biosynthesis of elastin or collagen.

In accordance with one aspect of the present invention, the above and other objects can be accomplished by the provision of a composition for promoting biosynthesis of elastin or collagen containing glycine, L-proline and a divalent metal ion, wherein the content ratio of glycine to L-proline is 1:0.9 to 1:1.1.

In accordance with another aspect of the present invention, there is provided a pharmaceutical composition for promoting the biosynthesis of elastin or collagen including the composition.

In accordance with another aspect of the present invention, there is provided a functional cosmetic composition for promoting the biosynthesis of elastin or collagen including the composition.

In accordance with another aspect of the present invention, there is provided a cosmetic product or a health functional food for preventing musculoskeletal diseases containing the composition for promoting the biosynthesis of elastin or collagen as an active ingredient.

In accordance with another aspect of the present invention, there is provided a method for promoting the biosynthesis of elastin or collagen including administering the composition.

In accordance with another aspect of the present invention, there is provided the use of the composition for enhancement of elastin or collagen biosynthesis.

In accordance with another aspect of the present invention, there is provided the use of the composition for the preparation of a drug for promoting elastin or collagen biosynthesis.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows the results of confirming the ability of the composition according to the present invention to grow fibroblasts (Hs27 cells).

FIG. 2 shows the results of qPCR confirming the change in the expression levels of COL1A2, ELN and COL4A1 genes in fibroblasts upon treatment with the composition according to the present invention.

FIG. 3 shows the results of Western blot confirming the production performance of type I collagen and elastin proteins in fibroblasts upon treatment with the composition according to the present invention.

### DETAILED DESCRIPTION OF THE INVENTION

Unless defined otherwise, all technical and scientific terms used herein have the same meanings as appreciated by those skilled in the field to which the present invention pertains. In general, the nomenclature used herein is well-known in the art and is ordinarily used.

In order to enhance the biosynthesis of collagen and elastin, which are components of dermal tissue, it is known that, when certain amino acids and oligopeptides are properly transported and applied locally or orally, gene expression causing the biosynthesis of dermal and epidermal connective tissues, especially collagen and tropoelastin, is promoted. However, research on an optimal amino acid combination that can promote the production of collagen and elastin in fibroblasts and a mineral composition supporting the same is insufficient. In the present invention, it was found that a composition containing glycine. L-proline and a divalent metal ion enhances the expression of genes involved in biosynthesis of collagen and elastin in fibroblasts, and promotes the production of collagen proteins and elastin proteins in fibroblasts.

In one aspect, the present invention is directed to a composition for promoting biosynthesis of elastin or collagen containing glycine. L-proline and a divalent metal ion, wherein a content ratio of glycine to L-proline is 1:0.9 to 1:1.1.

In the present invention, the content ratio of glycine to L-proline is preferably 1:0.9 to 1:1.1, more preferably 1:0.95 to 1:1.05, and most preferably 1:0.99 to 1:1.01.

The composition of the present invention may further contain one or more amino acids selected from the group consisting of L-alanine, L-valine. L-leucine and L-lysine hydrochloride, and the content ratio of each amino acid to glycine based on weight is preferably as follows:
L-alanine: 0.40 to 0.90,
L-valine: 0.30 to 0.80.
L-leucine: 0.10 to 0.30.
L-lysine hydrochloride: 0.10 to 0.25;
more preferably,
L-alanine: 0.60 to 0.85,
L-valine: 0.40 to 0.75,
L-leucine: 0.15 to 0.25,
L-lysine hydrochloride: 0.12 to 0.20; and

most preferably,
L-alanine: 0.70 to 0.80,
L-valine: 0.55 to 0.70,
L-leucine: 0.18 to 0.23.
L-lysine hydrochloride: 0.14 to 0.18.

The composition of the present invention may further contain 0.1 to 30.0% by weight, preferably 0.5 to 25.0% by weight, more preferably 1.0 to 20% by weight of L-cysteine or N-acetyl-L-cysteine based on the total amino acid composition.

The composition according to the present invention may have a total amino acid content of 1 to 700 g/L, preferably 5 to 650 g/L, more preferably 10 to 600 g/L, and most preferably 15 to 500 g/L, but is not limited thereto.

In the present invention, the divalent metal may be selected from the group consisting of copper (Cu), cobalt (Co), calcium (Ca), magnesium (Mg), manganese (Mn) and zinc (Zn). The divalent metal ion may be present in an amount of 0.001 to 5.0% by weight, preferably 0.003 to 4.5% by weight, and most preferably 0.005 to 4.0% by weight, based on the metal ion, with respect to the total composition, but is not limited thereto.

Preferably, the divalent metal ion present in the composition according to the present invention is copper. Copper may be present as a copper metal itself, but is preferably present in the form of a copper salt compound or a hydrate thereof, more preferably in the form of a copper sulfate hydrate, and most preferably in the form of copper sulfate pentahydrate, but is not limited thereto.

Copper (Cu) used in the composition of the present invention is an essential micro mineral present as a part of various proteins including enzymes in the body while conversion between two valence elements (Cu⁺ and Cu²⁺) takes place, like iron. Copper is involved in the production of ATP in the final process of the electron transport system in the mitochondria, and copper is bound to superoxide dismutase (SOD) and thus has an antioxidant function of preventing oxidative damage to cells and additionally functions to deposit a melanin pigment in the skin along with manganese and tyrosine (amino acids).

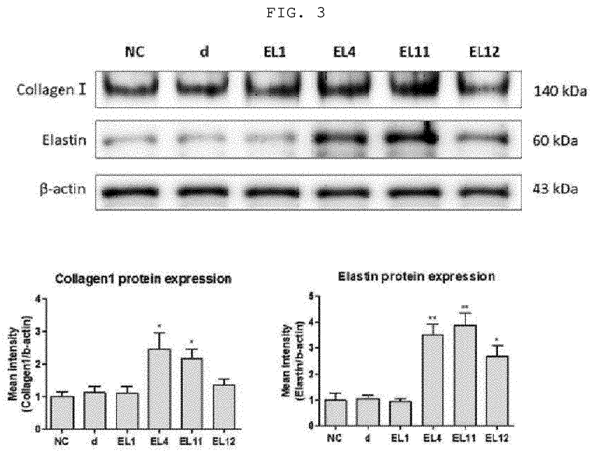

In one embodiment of the present invention, when treating fibroblasts (Hs27 cells) with the composition containing glycine, L-proline and copper, and detecting changes in the expression levels of the COL1A2 gene, which is a gene involved in type I collagen biosynthesis, an ELN gene, which is a gene involved in elastin biosynthesis, and the COL4A1 gene through qPCR, it was found that the expression of the COL1A2 gene and the ELN gene was increased (see FIG. 2).

In another embodiment of the present invention, fibroblasts (Hs27 cells) were treated with the composition containing glycine, L-proline and copper, and changes in the production of type I collagen and elastin were detected by Western blot. As a result, it was found that the production ability of the type I collagen and elastin protein increased (see FIG. 3).

In another aspect, the present invention is directed to a pharmaceutical composition for promoting the biosynthesis of elastin or collagen including the composition.

The pharmaceutical composition of the present invention can be used for the prevention or treatment of elastin-related diseases, hypertension caused by decreased vascular elasticity and decreased blood circulation, decreased tendon and ligament elasticity, decreased joint elasticity, elastic fibrosis due to photoaging, epidermal atrophy, dermal atrophic skin disease, burns, radiation burns, skin lesions, bed sores, dermal aplasia caused by drug administration, or muscle and joint lesions, but is not limited thereto.

In addition, the pharmaceutical composition of the present invention can be used for improvement of blood vessel elasticity by induction of elastin and/or collagen and treatment of diseases or amelioration in health conditions through the same.

The pharmaceutical composition of the present invention may be formulated as an oral administration, an intramuscular injection, a percutaneous injection, a medicine for external application, or a medical device for injection or external application.

The pharmaceutical composition according to the present invention also contains hyaluronic acid or a salt thereof, particularly hyaluronic acid or a salt thereof, preferably a hyaluronic acid sodium salt, having an average molecular weight of 500,000 to 3,000,000 Da in an amount of 0.01 to 3% by weight of the total composition.

The composition containing a mixture of the above-described amino acid and hyaluronic acid or a salt thereof is suitable for use in the form of oral administration, intramuscular injection, percutaneous injection, external medicine or a medical device, and may be formulated into a form appropriate therefor and used.

Non-limiting examples of formulations that can be used include injections, gels, ointments, emulsions, transdermal patches, sterile solutions, fillers, wound coatings and amino acid powders designed to be reconstituted with a sterile aqueous solution of hyaluronic acid or a salt thereof.

Formulations for injection are prepared by dissolving an amino acid in the form of a powder in a sterile solution of hyaluronic acid or a salt thereof, preferably by directly introducing the hyaluronic acid or a salt thereof, preferably, a sodium hyaluronate gel, into a vial containing a powder (e.g., using a skin graft syringe). After complete dissolution, the resulting gel solution is injected into the dermal site. The sterile aqueous solution of hyaluronic acid or a salt thereof may contain a pH-adjusting buffer (e.g. phosphate buffer) or an osmotic pressure adjusting agent (e.g. sodium chloride), and other technical adjuvants, which can ensure the physicochemical and tissue compatibility required for sterile injectable pharmaceutical formulations.

In another aspect, the present invention is directed to a cosmetic composition, in particular a functional cosmetic composition including the composition for promoting the biosynthesis of elastin or collagen according to the present invention.

The functional cosmetic composition according to the present invention may be used for improving skin elasticity and removing wrinkles and/or facial lines, but is not limited thereto.

Depending on the purpose of use and functions thereof, the cosmetic composition of the present invention can be formulated in various forms such as a solution (lotion-type composition), concentrated solution, gel, ointment, emulsion (cream, emulsion), vesicle dispersion, powder, dense powder, paste or solid, and can be provided as a functional cosmetic.

Specifically, the cosmetic containing the cosmetic composition according to the present invention includes all products including creams such as facial creams, hand creams, moisturizing creams and sun protection creams, solutions or concentrated solutions, lotions, cream powders, foundations, microemulsions, ointments, pastes, packs and sprays, but is not limited thereto.

When the composition of the present invention is used for improvement of elasticity and removal of wrinkles and facial lines, and as a moisturizing agent, the compositions for these applications are preferably provided in the form of emulsions such as creams, gels, lotions, ointments, or vesicle dispersions, and may further include other pharmaceutical or functional active ingredients.

The functional cosmetic composition may contain modified or unmodified oils of vegetable or animal oils. Examples of these oils include sweet almond oil, avocado oil, castor oil, olive oil, jojoba oil, sunflower oil, wheat flour oil, sesame oil, peanut oil, grapeseed oil, soy milk, safflower oil, coconut oil, maize oil, hazelnut oil, Karite butter, palm oil, apricot kernel oil, calophyllum oil, or perhydrosqualene. Moreover, the oil phase may be an inorganic oil such as liquid paraffin or liquid petrolatum. The oil may be a fatty acid ester such as isopropyl myristate, isopropyl palmitate, 2-ethylhexyl palmitate, penicillin oil (stearyl octonate), unsaturated fatty acid such as oleic acid, palmitic acid, stearic acid, behenic acid, linoleic acid and linolenic acid, or a volatile or nonvolatile isoparaffin such as fatty acid or C8-C16 isoparaffin. The oil may be a C12-C18 fatty alcohol such as oleyl alcohol, cetyl alcohol or stearyl alcohol.

When the composition is provided as an emulsion, the emulsified composition of the present invention contains an oil phase and an aqueous phase. The oil phase is preferably provided in the range of about 1 to about 75% by weight, more preferably about 5 to about 60% by weight, and most preferably about 40 to about 60% by weight, based on the total weight of the composition.

The aqueous phase contains an adjuvant commonly used in aqueous gels and cosmetic emulsions. The aqueous phase may be provided in an amount from about 0.5 to about 20% by weight, and may include lower C2-C6 mono-alcohols and/or polyols such as glycerol, butylene glycol, isoprene glycol, propylene glycol, ethylene glycol and the like.

The emulsifier can be used for the preparation of the emulsified cosmetic composition. A cosmetically acceptable emulsifier can be used in any amount as long as it exhibits the desired emulsion effect. The emulsifier is selected from known salts and surfactants. The emulsifier is preferably selected from stearic acid, sorbitan sesquioleate, polyethylene glycol (PEG-30), dipolyhydroxystearate, lecithin, magnesium stearate, and derivatives and mixtures thereof. The emulsifier is preferably provided in a range of about 0.5 to about 30% by weight based on the total weight of the composition, more preferably about 1 to about 12% by weight, and more preferably about 4 to about 8% by weight.

In another aspect, the present invention is directed to a health functional food containing the composition for promoting biosynthesis of elastin or collagen according to the present invention as an active ingredient.

The functional food according to the present invention can be used for skin health, improvement in blood circulation, blood pressure control, or prevention and treatment of musculoskeletal diseases including joint diseases, specifically improvement in skin elasticity, removal of wrinkles, removal of facial lines, alleviation of hypertension, improvement in blood circulation, improvement of elasticity of tendons and ligaments, improvement of joint elasticity, and prevention or treatment of elastic fibrosis due to photoaging, dermal epidermal atrophy, dermal atrophic skin disease, burns, radiation burns, skin lesions, bed sores, and dermal aplasia or muscle and joint lesions caused by drug administration, and is preferably used for cosmetic application or joint ailment prevention.

In addition, the functional food of the present invention can be utilized in various applications including drugs, foods and beverages for preventing oxidation. The functional food of the present invention may be used as, for example, various foods, candy, chocolate, beverages, gum, tea, vitamin complexes, health supplements and the like, and may be used in the form of powders, granules, tablets, capsules or beverages.

The composition of the present invention includes various nutrients, vitamins, minerals (electrolytes), flavoring agents such as synthetic flavoring agents and natural flavoring agents, coloring agents and fillers (cheese, chocolate, etc.), pectic acid and salts thereof, alginic acid and salts thereof, and organic acids, protective colloid thickeners, pH adjusters, stabilizers, preservatives, glycerin, alcohols, carbonation agents used in carbonated beverages, and the like. In addition, the composition of the present invention may contain flesh for the production of natural fruit juice, fruit juice beverages and vegetable beverages. These ingredients may be used independently or in combination.

In another aspect, the present invention is directed to a method for promoting biosynthesis of elastin or collagen including administering the composition.

In another aspect, the present invention is directed to the use of the composition for enhancement of elastin or collagen biosynthesis.

In another aspect, the present invention is directed to the use of the composition for the preparation of a drug for promoting elastin or collagen biosynthesis.

### EXAMPLE

Hereinafter, the present invention will be described in more detail with reference to examples. However, it will be obvious to those skilled in the art that these examples are provided only for illustration of the present invention and should not be construed as limiting the scope of the present invention.

### Example 1: Confirmation of Toxicity to Fibroblasts of Composition According to Present Invention

The composition according to the present invention was prepared as shown in Table 1 below, and the cytotoxicity to fibroblasts of the composition of Table 1 was determined.

The fibroblasts herein used were human-derived fibroblasts Hs27 (ATCC, #CRL 1634) and the cells were cultured in DMEM (Dulbecco's Modified Eagle Medium) containing 10% FBS (fetal bovine serum) and 1% penicillin-streptomycin.

A drug prepared in a dose of 1000 μg/10 μl was diluted in serum-free DMEM medium containing 1% penicillin-streptomycin for treatment of cells.

TABLE 1

| | Content of each ingredient in formulation (μg/10 μl) | | | | |
|---|---|---|---|---|---|
| Ingredient | D | EL1 | EL4 | EL11 | EL12 |
| Glycine | 302 | 266.7 | 266.7 | 266.7 | 266.7 |
| L-proline | 227 | 266.7 | 266.7 | 266.7 | 266.7 |
| L-alanine | 228 | 200 | 200 | 200 | 200 |
| L-valine | 168 | 166.7 | 166.7 | 166.7 | 166.7 |
| L-leucine | 42 | 56.7 | 56.7 | 56.7 | 56.7 |
| L-lysine HCl | 33 | 43.3 | 43.3 | 43.3 | 43.3 |
| Sum | 1000 | 1000.1 | 1000.1 | 1000.1 | 1000.1 |
| Copper sulfate | 0 | 0 | 3.3 | 1.67 | 0.33 |
| pentahydrate | | | | | |
| Total | 1000 | 1000.1 | 1003.4 | 1001.77 | 1000.43 |

1×10⁵ Hs27 cells were seeded in a 48-well plate and cultured for 24 hours. After 24 hours, the cells were treated with the drug prepared above. In order to observe cytotoxicity, an EZ-cytox (Dogen Bio, Korea) kit for measuring cell viability was used after 24 hours, 48 hours and 72 hours. Water soluble tetrazolium salts (WST) that reacted with enzymes present in viable cells contained in KIT was treated, absorbance at 450 nm was measured, and cell viability was calculated.

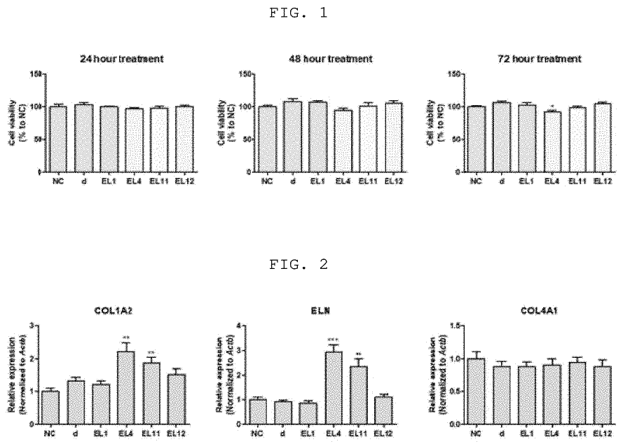

As a result, as shown in FIG. 1, compared to the NC group treated only with the solvent, it was found that the cells treated with d, EL1, EL11 and EL12 did not show cytotoxicity even after 24 hours, 48 hours or 72 hours. It was found that the cells treated with EL4 did not show toxicity after 24 hours or 48 hours, but the cell viability thereof decreased slightly after 72 hours.

### Example 2: Determination of COL1A2, COL4A1 and ELN Gene Expression Levels Upon Treatment with Composition According to Present Invention

Changes in the expression levels of genes involved in the biosynthesis of collagen and elastin in Hs27 cells according to the treatment of the composition according to the present invention were determined.

5×10⁶ Hs27 cells were seeded in a 100π dish and cultured for 24 hours. The cells were treated with the prepared compositions (d, EL1, EL11 and EL12) after 24 hours. RNA was extracted using TRIzol after 72 hours to determine the gene expression level. 1 μg of RNA extracted from each sample was synthesized into cDNA using a cDNA reverse transcriptase, and the expression levels of COL1A2, ELN, COL4A1 and ACTB genes were detected using a real-time PCR detection system (Biorad CFX Connect). In order to identify each gene, the primers of Table 2 were used, and relative expression levels were calculated using ACTB (β-actin) as an internal control.

TABLE 2
Primer sequences for detecting gene expression
level

| Gene | Forward | Reverse |
|---|---|---|
| COL1A2 | CCA AAT CTG TCT CCC | TCA AAA ACG AAG GGG |
| | CAG AA (SEQ ID NO: 1) | AGA TG |
| | | (SEQ ID NO: 2) |
| ELN | CCA TGT CCA CAC AAG | GCC AGA GTG GCT TTC |
| | GAC AG (SEQ ID NO: 3) | TCA AC |
| | | (SEQ ID NO: 4) |
| COL4A1 | GCT TGA AAA GGG TTG | TTG AGT CCC GGT AGA |
| | AGC AG (SEQ ID NO: 5) | CCA AC |
| | | (SEQ ID NO: 6) |
| ACTB | GAG GCC TGG ACT CTC | AAT GAA TGG GGG TTG |
| | AAC TG (SEQ ID NO: 7) | AAT GA |
| | | (SEQ ID NO: 8) |

As a result, as shown in FIG. 2, compared to the NC group treated only with solvent, it was found that there was no significant change in the expression levels of the COL1A2 gene and the ELN gene in the cells treated with the composition d, EL1 and EL12 after 72 hours, and it was found that the expression levels of the COL1A2 gene and ELN gene in the cell groups treated with EL4 and EL11 were significantly increased after 72 hours. In addition, unlike the COL1A2 gene and the ELN gene, it was found that there was no change in the expression level in the COL4A1 gene, under all composition treatment conditions.

### Example 3: Confirmation of Protein Production Ability of COL1A2, COL4A1, ELN Upon Treatment with Composition According to Present Invention

Changes in the ability to produce collagen and elastin proteins in Hs27 cells upon treatment with the composition according to the present invention were detected.

5×10⁶ Hs27 cells were seeded in a 100π dish and cultured for 24 hours. The cells were treated with the prepared compositions (d, EL1, EL11 and EL12) after 24 hours. To detect the protein expression level, after 72 hours, the protein was extracted using a protein lysis buffer (cell signaling, #9803). Western blotting was performed on each protein by the following method to identify type I collagen, elastin and D-actin proteins.

Type I collagen: Western blot was performed in a native environment (non-denaturing condition) to determine the protein expression level of type I collagen. A sample was prepared by diluting 30 μg of the extracted protein in a native sample buffer (Biorad, #1610738).

Electrophoresis was performed using an 8% acrylamide gel containing no SDS and a tank buffer (Tris-glycine buffer), and the protein separated on the gel was transferred to the PVDF membrane and then blocked with skim milk for 2 hours. After that, the protein was allowed to stand at 4° C. overnight using an anti-collagen I antibody (abcam, ab34710) and was then reacted with a secondary antibody for 1 hour. The reaction of the antibody was detected using an ECL solution and an Amersham Imager 600 imaging system.

Elastin and β-actin: Western blot was performed in a general Western blot environment (non-native and denaturing conditions) to detect the protein expression levels of elastin and β-actin. A sample was prepared by diluting 30 μg of the extracted protein in a 4× Laemmli sample buffer (Biorad, #1610747). Electrophoresis was performed using 10% acrylamide gel containing SDS and a tank buffer (Tris-glycine buffer). Then, the protein separated on the gel was transferred to the PVDF membrane and blocked using skim milk for 2 hours. Then, the protein was allowed to stand at 4° C. overnight using Anti-Elastin antibody (Santacruz, sc-166543) and Anti-β-actin antibody (Santacruz, sc-47778) and then reacted with a secondary antibody for 1 hour. The reaction of the antibody was detected using an ECL solution and an Amersham Imager 600 imaging system.

As a result, as shown in FIG. 3, compared to the NC group treated with a solvent, the cells treated with the d, EL1 and EL12 compositions were found to show an increase in the protein expression level of type I collagen after 72 hours. Elastin was not changed in the groups treated with d and EL1, but was significantly increased in the groups treated with the EL4, EL11 and EL12 compositions.

### INDUSTRIAL AVAILABILITY

The composition according to the present invention can enhance the biosynthesis of elastin and collagen of fibroblasts and is thus useful for restoring skin elasticity deteriorated due to aging or exposure to light and for removing wrinkles and facial lines.

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

1. A composition for promoting biosynthesis of elastin or collagen comprising glycine, L-proline and a divalent metal ion, wherein a content ratio of glycine to L-proline is 1:0.9 to 1:1.1.

2. The composition according to claim 1, wherein the composition further comprises one or more amino acids selected from the group consisting of L-alanine, L-valine, L-leucine and L-lysine hydrochloride, and a content ratio of each amino acid to glycine based on weight is as follows:
L-alanine: 0.40 to 0.90;
L-valine: 0.30 to 0.80;
L-leucine: 0.10 to 0.30;
L-lysine hydrochloride: 0.10 to 0.25.

3. The composition according to claim 1, wherein the composition further comprises 0.1 to 30.0% by weight of L-cysteine or N-acetyl-L-cysteine.

4. The composition according to claim 1, wherein the divalent metal is selected from the group consisting of copper (Cu), cobalt (Co), calcium (Ca), magnesium (Mg), manganese (Mn) and zinc (Zn).

5. The composition according to claim 4, wherein the divalent metal is copper (Cu).

6. The composition according to claim 4, wherein the divalent metal is present in an amount of 0.001 to 5.0% by weight based on a total weight of the composition.

7. The composition according to claim 1, wherein the composition further comprises hyaluronic acid or a salt thereof.

8. A pharmaceutical composition for promoting biosynthesis of elastin or collagen comprising the composition according to claim 1.

9. The pharmaceutical composition according to claim 8, wherein the pharmaceutical composition is formulated as an oral administration form, an intramuscular injection form, a percutaneous injection form, an external medicine form or a medical device.

10. The pharmaceutical composition according to claim 8, wherein the pharmaceutical composition is used for prevention or treatment of elastin-related diseases, hypertension due to decreased vascular elasticity, decreased blood circulation, decreased tendon and ligament elasticity, decreased joint elasticity, elastic fibrosis due to photoaging, dermal epidermal atrophy, dermal atrophic skin disease, burns, radiation burns, skin lesions, bed sores, dermal aplasia caused by drug administration, or muscle and joint lesions.

11. A functional cosmetic composition for promoting biosynthesis of elastin or collagen comprising the composition according to claim 1.

12. A functional cosmetic comprising the functional cosmetic composition according to claim 11.

13. The functional cosmetic according to claim 12, wherein the functional cosmetic is used for improving skin elasticity and removing wrinkles and/or facial lines.

14. The functional cosmetic according to claim 12, wherein the functional cosmetic is provided in a form of a cream including a facial cream, a hand cream, a moisturizing cream or a sun protection cream, a solution or concentrated solution including a lotion, a cream powder, a foundation, a microemulsion, an ointment, a paste, a pack or a spray.

15. A health functional food beneficial for improving skin health, blood circulation, blood pressure control, or musculoskeletal system health, including joints, comprising the composition according to claim 1.
